(19) United States
(12) Patent Application Publication
(10) Pub. No.: US 2026/0098256 A1
(43) Pub. Date: Apr. 9, 2026

(54) Methods of Improving Unique Molecular Index Ligation Efficiency

(71) Applicant: Illumina, Inc., San Diego, CA (US)

(72) Inventors: Louise Fraser, Great Cambourne (GB); Natalie Morrell, Cambridge (GB); Ana Rita Borba, Cambridge (GB); Niall Anthony Gormley, Cambridge (GB); Ming-Hsiang Lee, San Diego, CA (US); Sorena Rahmanian, San Diego, CA (US); Jennifer Lococo, San Diego, CA (US); Danny Chou, San Diego, CA (US)

(73) Assignee: Illumina, Inc., San Diego, CA (US)

(21) Appl. No.: 19/417,801

(22) Filed: Dec. 12, 2025

Related U.S. Application Data
(63) Continuation of application No. PCT/US24/57746, filed on Nov. 27, 2024.
(60) Provisional application No. 63/603,245, filed on Nov. 28, 2023.

(51) Int. Cl. C12N 15/10

(52) U.S. Cl. CPC C12N 15/1065

(57) ABSTRACT

The disclosed embodiments concern methods for determining sequences of interest using improved unique molecular indexes (UMIs) that are uniquely associable with individual polynucleotide fragments. Pools of adapters comprising UMIs are also provided.

This application is a bypass continuation of PCT/US2024/057746, filed Nov. 27, 2024, which claims priority to U.S. Provisional Application No. 63/603,245, filed Nov. 28, 2023, which is incorporated herein in its entirety for any purpose.

### FIELD

This disclosure relates to methods, apparatuses, systems, and compositions for improving unique molecular index (UMI) ligation efficiency.

### BACKGROUND

Next generation sequencing (NGS) technology is providing increasingly high throughput of sequencing at low cost, allowing larger sequencing depth. However, because sequencing accuracy and sensitivity are affected by errors and noise from various sources, e.g., sample defects, PCR during library preparation, enrichment, clustering, and sequencing, increasing depth of sequencing alone cannot ensure detection of sequences of very low allele frequency, such as in fetal cell-free DNA (cfDNA) in maternal plasma, circulating tumor DNA (ctDNA), and sub-clonal mutations in pathogens. For example, analysis of cell free DNA (cfDNA) can be used to detect somatic variants in blood without the need for biopsy; however, the low percentage of circulating tumor DNA (ctDNA) within total cfDNA causes variant allele frequencies to exist near the limit of detection of existing methods. Artifacts that may arise from library preparation methods can be mistaken as low frequency variants, thereby decreasing the sensitivity and reliability of the methods. Therefore, it is desirable to develop methods for determining sequences of DNA molecules in small quantity and/or low allele frequency while suppressing sequencing inaccuracy due to various sources of errors.

Libraries prepared according to ligation-based technologies can be improved by incorporation of Unique Molecular Identifiers (UMIs) to lower the rate of inherent errors in NGS data. However, adapters comprising UMIs do not always ligate properly to source nucleic acid molecules and, thus, there is a need to increase ligation efficiency of adapter to DNA. Library conversion efficiency could be enhanced using adapters with improved UMI sequences in ligation-based preparations. The present disclosure provides materials and methods for preparing improved UMIs.

### SUMMARY

In accordance with the description, described herein are methods and compositions using unique molecular indices (UMIs).

Embodiment 1. A method for sequencing source nucleic acid molecules from a sample using a pool of adapters, wherein the adapters each comprise a unique molecular identifier (UMI), wherein each UMI comprises an oligonucleotide sequence comprising the sequence 5′-X_{N}YZ-3′, wherein the 3′ end of the UMI ends with YZ, wherein YZ does not correspond to GA, GC, or GG, wherein N is an integer greater than or equal to 1 and each X is the same or different, wherein each adapter is attached to a solid support, and wherein each UMI is an oligonucleotide sequence that can be used to identify an individual molecule of a source nucleic acid fragment in the sample, comprising: (a) fragmenting source nucleic acid molecules into nucleic acid fragments: (b) applying the pool of adapters to source nucleic acid fragments in the sample: (c) amplifying both strands of the source nucleic acid-adapter products to obtain a plurality of amplified polynucleotides: (d) sequencing the plurality of amplified polynucleotides, thereby obtaining a plurality of reads each associated with a UMI; (e) identifying a plurality of UMIs associated with the plurality of reads: (f) determining sequences of the source nucleic acid fragments in the sample using the plurality of reads obtained in (c) and the plurality of UMIs identified in (d).

Embodiment 2. A method for sequencing source nucleic acid molecules from a sample using a pool of adapters, wherein the adapters each comprise a primer, a primer binding site, an index sequence, and/or a barcode, wherein each adapter comprises an oligonucleotide sequence comprising the sequence 5′-X_{N}YZ-3′, wherein the 3′ end of the adapter ends with YZ, wherein YZ does not correspond to GA, GC, or GG, wherein N is an integer greater than or equal to 1 and each X is the same or different, and wherein each adapter is attached to a solid support, comprising: (a) fragmenting source nucleic acid molecules into nucleic acid fragments; (b) applying the pool of adapters to source nucleic acid fragments in the sample; (c) amplifying both strands of the source nucleic acid-adapter products to obtain a plurality of amplified polynucleotides; and (d) sequencing the plurality of amplified polynucleotides.

Embodiment 3. The method of embodiment 1 or 2, wherein the source nucleic acid is double-stranded.

Embodiment 4. The method of embodiment 3, wherein the double-stranded source nucleic acids are double-stranded DNA.

Embodiment 5. The method of embodiment 3, wherein the double-stranded source nucleic acids are ctDNA.

Embodiment 6. The method of embodiment 3, wherein the double-stranded source nucleic acids are cfDNA.

Embodiment 7. The method of embodiment 3, wherein the double-stranded source nucleic acids are RNA.

Embodiment 8. The method of embodiments 3-7, wherein the adapters each comprise a unique molecular identifier (UMI), wherein a first UMI is on the first strand of the double-stranded source nucleic acid fragments.

Embodiment 9. The method of embodiment 8, wherein the adapters each comprise a unique molecular identifier (UMI), wherein a first copy of the first UMI is on the first strand and a second copy of the first UMI is on the second strand of the double-stranded source nucleic acid fragments.

Embodiment 10. The method of any one of embodiments 3-9, wherein the adapters each comprise a unique molecular identifier (UMI), wherein a first UMI is on a first strand of the double-stranded source nucleic acid fragments, and a second UMI is on the second strand of the double-stranded source nucleic acid fragments.

Embodiment 11. A method of sequencing a double-stranded nucleic acid library produced by the method of any one of embodiments 1-10, wherein the adapters each comprise a unique molecular identifier (UMI), and wherein the UMIs are sequenced to provide increased sensitivity in DNA sequencing.

Embodiment 12. A pool of adapters, wherein the adapters each comprise a unique molecular identifier (UMI), wherein each UMI comprises an oligonucleotide sequence comprising the sequence 5′-X_{N}YZ-3′, wherein the 3′ end of the UMI ends with YZ, wherein YZ does not correspond to GA, GC, or GG, wherein N is an integer greater than or equal to 1 and each X is the same or different, and wherein each adapter is attached to a solid support.

Embodiment 13. A pool of adapters, wherein the adapters each comprise a primer, a primer binding site, an index sequence, and/or a barcode, wherein each adapter comprises an oligonucleotide sequence comprising the sequence 5′-X_{N}YZ-3′, wherein the 3′ end of the adapter ends with YZ, wherein YZ does not correspond to GA, GC, or GG, wherein N is an integer greater than or equal to 1 and each X is the same or different, and wherein each adapter is attached to a solid support.

Embodiment 14. The pool of adapters of embodiment 12 or 13, wherein each N is an integer from 1 to 40, 1 to 30, 1 to 20, or 1 to 10.

Embodiment 15. The pool of adapters of any one of embodiments 12-14, wherein each N is 2, 3, 4, 5, 6, 7, 8, 9, 10, 11, 12, 13, 14, 15, 16, 17, 18, 19, 20, 21, 22, 23, 24, 25, 26, 27, 28, 29 30, 31, 32, 33, 34, 35, 36, 37, 38, 39, or 40.

Embodiment 16. The pool of adapters of any one of embodiments 12-15, wherein each N is an integer from 2 to 7.

Embodiment 17. The pool of adapters of any one of embodiments 12-15, wherein each N is an integer from 4 to 7.

Embodiment 18. The pool of adapters of any one of embodiments 12-15, wherein each N is an integer from 4 to 5.

Embodiment 19. The pool of adapters of any one of embodiments 12-15, wherein each N is an integer from 6 to 7.

Embodiment 20. The pool of adapters of any one of embodiments 12-19, wherein YZ corresponds to TX.

Embodiment 21. The pool of adapters of any one of embodiments 12-20, wherein YZ corresponds to TA, TC or TG.

Embodiment 22. The pool of adapters of any one of embodiments 12-21, wherein the solid support is a flowcell.

Embodiment 23. The pool of adapters of any one of embodiments 12-22, wherein the adapters comprise DNA.

Embodiment 24. The pool of adapters of any one of embodiments 12-23, wherein the adapters comprise RNA.

Embodiment 25. The pool of adapters of any one of embodiments 12-24, wherein the adapters comprise an RNA: DNA hybrid.

Embodiment 26. The pool of adapters of any one of embodiments 12-25, wherein the adapters are methylated.

Embodiment 27. The pool of adapters of any one of embodiments 12-26, wherein the adapters are single stranded.

Embodiment 28. The pool of adapters of any one of embodiments 12-26, wherein the adapters are double stranded.

Embodiment 29. The pool of adapters of embodiment 28, wherein the double stranded adapters each comprise a UMI, wherein the UMI is on only one strand.

Embodiment 30. The pool of adapters of embodiment 28, wherein the double stranded adapters each comprise a UMI, wherein the UMI is on both strands.

Embodiment 31. The pool of adapters of any one of embodiments 12-30, wherein each adapter in the pool of adapters comprises a UMI and wherein the UMI is a unique UMI shared by no other adapter in the pool of adapters.

Embodiment 32. The pool of adapters of any one of embodiments 12-30, wherein each adapter in the pool of adapters comprises a UMI and wherein more than one adapter in the pool of adapters has the same UMI, but wherein that UMI differs from other adapters in the pool of adapters.

Embodiment 33. The method of any one of embodiments 1-11 or the pool of adapters of any one of embodiments 12-32, wherein the adapters each comprise a primer.

Embodiment 34. The method of any one of embodiments 1-11 or 33 or the pool of adapters of any one of embodiments 12-33, wherein the adapters each comprise a primer binding site.

Embodiment 35. The method of any one of embodiments 1-11 or 33-34 or the pool of adapters of any one of embodiments 12-34, wherein the adapters each comprise an index sequence.

Embodiment 36. The method of any one of embodiments 1-11 or 33-35 or the pool of adapters of any one of embodiments 12-35, wherein the adapters each comprise a barcode.

Embodiment 37. The method of any one of embodiments 1-11, wherein the method uses the pool of adapters of any one of claims 12-36.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

### BRIEF DESCRIPTION OF THE DRAWINGS

FIG. 1A provides an overview of the library preparation protocol, showing that adapter ligation has variable efficiency. FIG. 1B depicts a schematic of a forked adapter.

FIGS. 2A-2B provides knee plots for 3′ UMI read counts (FIG. 2A) and 5′UMI read counts (FIG. 2B) FIG. 2 shows the variability of different UMIs across over 100 sequences in terms of their representation at sequencing.

FIGS. 3A-3B provide graphs of UMI functional testing broken down by UMI subgroups for three different samples (40 Regular (R40), 40 Good (G40), and 40 Poor (P40) performing UMIs). FIG. 3A depicts median exon coverage, and FIG. 3B shows median family size.

FIG. 4A provides a schematic depicting ligation of adapters with fragmented source DNA. FIG. 4B provides a graph quantifying cfDNA with adapters across different UMI subgroups. UMI subgroups included 40 Regular (R40), 40 Good (G40), and 40 Poor (P40) performing UMIs.

FIG. 5 provides a schematic showing elongated adapter.

FIG. 6 provides a graph depicting library yield of the various UMIs including the improved UMIs which were extended with -TG and -TC. G3=3 top performers, P3=3 bottom performers, P3+TG=3 bottom performers extended with TG, P3+TC=3 bottom performers extended with TC.

FIG. 7 provides the data of FIG. 6, plotted according to specific UMIs. G3=3 top performers, P3=3 bottom performers, P3+TG=3 bottom performers extended with TG, P3+TC=3 bottom performers extended with TC.

FIG. 8 provides a plot depicting the relative composition of library according to size (bp).

FIGS. 9A-9B provide similar plots of library content of Good UMIs (FIG. 9A) and Poor UMIs (FIG. 9B). The arrow indicates that good UMIs formed more adapter dimers than poor UMIs.

FIGS. 10A-C provides the data of FIG. 9B, plotted according to specific Poor UMIs.

FIG. 11 shows data for the adapter peak, adapter dimer peak and library peaks for the pool of 120 UMIs (120 UMI), the 3 top performing UMIs (G3), the 3 bottom performing UMIs (P3) and the 3 bottom performing UMIs extended with TG (P3+TG) or TC (P3+TC).

### DESCRIPTION OF THE EMBODIMENTS

### I. Compositions

Described herein are adapters, used in NGS sequencing methods. Such adapters each comprise a unique molecular identifier (UMI) used to identify individual nucleic acid molecules generated from source nucleic acids. This, as discussed below, helps investigators identify errors in sequencing generated from amplification, library preparation, etc. Improved UMI sequences facilitate ligation of adapters to source nucleic acids.

Also described herein are solid supports, such as flowcells, or beads which may be used to immobilize oligonucleotides described herein.

### A. Unique Molecular Identifiers

Unique molecular indices (UMIs) are sequences of nucleotides applied to or identified in RNA or DNA molecules that may be used to distinguish individual nucleic acid molecules from one another. Since UMIs are used to identify nucleic acid molecules, they are also referred to as unique molecular identifiers. See, e.g., Kivioja, Nature Methods 9, 72-74 (2012). UMIs may be sequenced along with the nucleic acid molecules with which they are associated to determine whether the read sequences are those of one source nucleic acid molecule or another. The term “UMI” is used herein to refer to both the sequence information of a polynucleotide and the physical polynucleotide per se. The term “UMI” can refer to both a “unique molecular identifier” and a “unique molecular index,” however, unique molecular identifiers are specifically used to identify nucleic acid molecules.

Commonly, multiple copies generated from a single source nucleic acid molecule are sequenced. In the case of sequencing by synthesis using Illumina's sequencing technology, the source nucleic acid molecule may be PCR amplified before delivery to a flow cell. Whether or not PCR amplified, in some instances, the individual DNA molecules applied to flow cell are bridge amplified or ExAmp amplified to produce a cluster. Each molecule in a cluster derives from the same source nucleic acid molecule but is separately sequenced. For error correction and other purposes, it can be important to determine that all reads from a single cluster are identified as deriving from the same source nucleic acid molecule. UMIs allow this grouping. The approach described herein may be used irrespective of which amplification method or sequencing method a user employs to sequence multiple copies generated from a single source nucleic acid molecule. A nucleic acid molecule that is copied by amplification or otherwise to produce multiple instances of the nucleic acid molecule is referred to as a source nucleic acid molecule. Types of source nucleic molecules include both RNA and DNA.

In addition to errors caused by amplifying the source nucleic acid molecules, errors can also occur in a region associated with the UMIs. In some implementations, the latter type of error may be corrected by mapping a read sequence to a most likely UMI among a pool of UMIs.

UMIs are similar to barcodes, which are commonly used to distinguish reads of one sample from reads of other samples, but UMIs are instead used to distinguish one source nucleic acid molecule from another when many nucleic acid molecules are sequenced together. Because there may be many more nucleic acid molecules in a sample than samples in a sequencing run, there are typically many more distinct UMIs than distinct barcodes in a sequencing run.

As mentioned, UMIs may be applied to or identified in individual source nucleic acid molecules. In some implementations, the UMIs may be applied to the source nucleic acid molecules by methods that physically link or bond the UMIs to the source nucleic acid molecules, e.g., by ligation or transposition through polymerase, endonuclease, transposases, etc. These “applied” UMIs are therefore also referred to as physical UMIs. In some contexts, they may also be referred to as exogenous UMIs. The UMIs identified within source nucleic acid molecules are referred to as virtual UMIs. In some contexts, virtual UMIs may also be referred to as endogenous UMI.

Physical UMIs may be defined in many ways. For example, they may be random, pseudo-random or partially random, or nonrandom nucleotide sequences that are inserted in adapters or otherwise incorporated in source nucleic acid molecules to be sequenced. In some implementations, the physical UMIs may be unique such that each can identify any given source nucleic acid molecule present in a sample without needing to consider any other information. The collection of adapters is generated, each having a physical UMI, and those adapters are attached to fragments or other source nucleic acid molecules to be sequenced, and the individual sequenced molecules each has a UMI that helps distinguish it from all other fragments. In such implementations, a very large number of different physical UMIs (e.g., many thousands to millions) may be used to uniquely identify DNA fragments in a sample.

A physical UMI must have a sufficient length to ensure this uniqueness for each and every source nucleic acid molecule. In some implementations, a smaller number of UMIs can be used than the number of source nucleic acid molecules in a sample, leading to less than a 1:1 correspondence between UMIs and source nucleic acid molecules. In this situation, a unique molecular identifier common to more than one source nucleic acid molecule can be used in conjunction with other identification techniques to ensure that each source nucleic acid molecule is uniquely identified during the sequencing process. In such implementations, multiple fragments or adapters may have the same physical UMI. Other information such as alignment location or virtual UMIs may be combined with the physical UMI to uniquely identify reads as being derived from a single source nucleic acid molecule/fragment. In some implementations, adapters include physical UMIs limited to a relatively small number of nonrandom sequences, e.g., 120) nonrandom sequences. Such physical UMIs are also referred to as nonrandom UMIs. In some implementations, the nonrandom UMIs may be combined with sequence position information, and/or virtual UMIs to identify reads attributable to a same source nucleic acid molecule. The identified reads may be combined to obtain a consensus sequence that reflects the sequence of the source nucleic acid molecule as described herein. Using physical UMIs, virtual UMIs, and/or alignment locations, one can identify reads having the same or related UMIs or locations, which identified reads can then be combined to obtain one or more consensus sequences. The process for combining reads to obtain a consensus sequence is also referred to as “collapsing” reads, which is further described hereinafter.

A “virtual unique molecular index” or “virtual UMI” is a unique subsequence in a source nucleic acid molecule. In some implementations, virtual UMIs are located at or near the ends of the source nucleic acid molecule. One or more such unique end positions may alone or in conjunction with other information uniquely identify a source nucleic acid molecule. Depending on the number of distinct source nucleic acid molecules and the number of nucleotides in the virtual UMI, one or more virtual UMIs can uniquely identify source nucleic acid molecules in a sample. In some cases, a combination of two virtual unique molecular identifiers is required to identify a source nucleic acid molecule. Such combinations may be extremely rare, possibly found only once in a sample. In some cases, one or more virtual UMIs in combination with one or more physical UMIs and/or locations may together uniquely identify a source nucleic acid molecule.

A “random UMI” may be considered a physical UMI selected as a random sample from a set of UMIs having of all possible different oligonucleotide sequences given one or more sequence lengths.

Conversely, a “nonrandom UMI” (NRUMI) as used herein refers to a physical UMI that is not a random UMI. In some embodiments, nonrandom UMIs are predefined for a particular experiment or application. In certain embodiments, rules are used to generate sequences for a set or select a sample from the set to obtain a nonrandom UMI. For instance, the sequences of a set may be generated such that the sequences have a particular pattern or patterns. In some implementations, each sequence differs from every other sequence in the set by a particular number of (e.g., 2, 3, or 4) nucleotides. That is, no nonrandom UMI sequence can be converted to any other available nonrandom UMI sequence by replacing fewer than the particular number of nucleotides. In some implementations, a set of NRUMIs used in a sequencing process includes fewer than all possible UMIs given a particular sequence length. In some implementations, nonrandom UMI information may be combined with other information, such as virtual UMIs, read locations on a reference sequence, and/or sequence information of reads, to identify sequence reads deriving from the same source nucleic acid molecule.

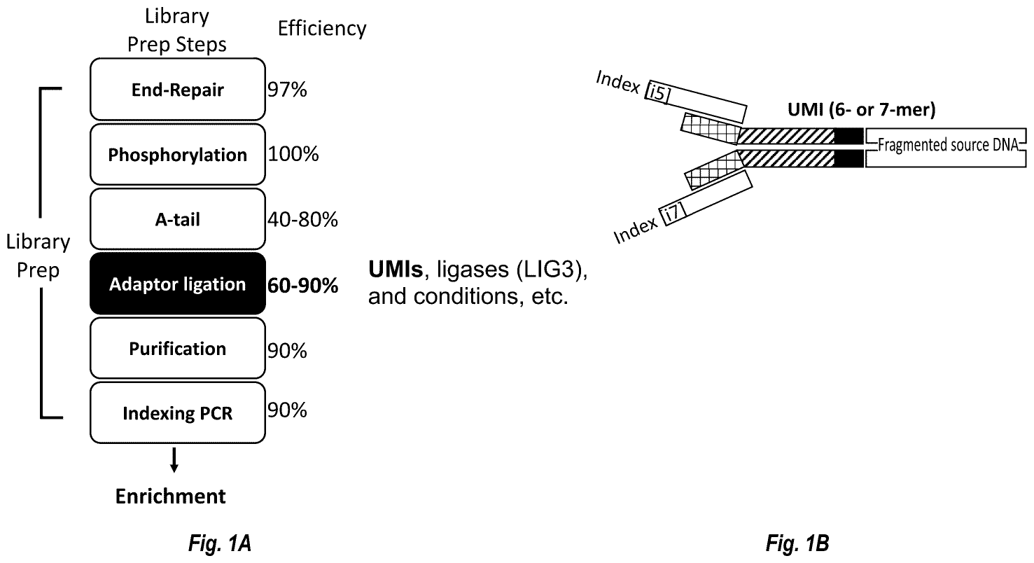

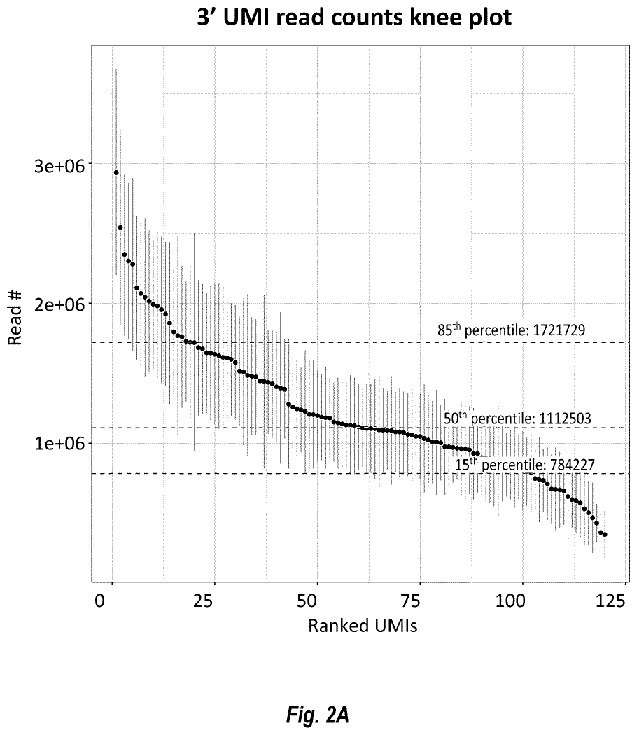

In certain embodiments, one UMI can be located on each strand of a double stranded source nucleic acid molecule as shown in FIG. 1B. In some implementations, an adapter has a duplex UMI in the double stranded region of the adapter, and each read includes a first UMI on one end of a fragmented source nucleic acid and a second UMI on the other end of the fragmented source nucleic acid.

The term “molecular length” is also referred to as sequence length and can be measured in nucleotides. The term molecular length is also used interchangeably with the terms molecular size, DNA size, and sequence length.

### B. Adapters

Described herein are a pool of adapters, having an improved sequence. In some embodiments the adapters each comprise a unique molecular identifier (UMI) having an improved sequence. As used herein, a “pool” may be referred to as a set or plurality of oligonucleotide species, for example, adapters. All sequences in a pool comprise a common feature. Methods and compositions herein may include more than one pool of adapters (e.g., a first pool of adapters and a second pool of adapters, etc.). In some embodiments, the methods and compositions herein are used with 2, 3, 4, 5, 6, 7, 8, 9, 10 or more pools of adapters. In such instances, oligonucleotides in a first pool may share a common feature and oligonucleotides in a second pool may share a different common feature, etc. A common feature in a pool may include a functional component, particular domain, and/or a particular modification.

In some embodiments, Unique Molecular Identifiers (UMIs) are used for quality control and can help identify rare variants, detect differential amplification, and/or enable the user to screen out probable sequencing errors.

As used herein, the term “adapter” refers generally to any linear oligonucleotide that can be ligated to a nucleic acid molecule of the disclosure. In some embodiments, adapters include two reverse complementary oligonucleotides forming a double-stranded structure. In some embodiments, an adapter includes two oligonucleotides that are complementary at one portion and mismatched at another portion, forming a Y-shape or fork-shaped adapter that is double stranded at the complementary portion and has two floppy overhangs at the mismatched portion. In some embodiments, adapters may comprise a primer, a primer binding site, an index sequence, a barcode, or any combination thereof. In some embodiments, each adapter is attached to a solid support.

In some embodiments, the adapters each comprise a primer. As used herein, a “primer” refers to an isolated oligonucleotide that is capable of acting as a point of initiation of synthesis when placed under conditions inductive to synthesis of an extension product (e.g., the conditions include nucleotides, an inducing agent such as DNA polymerase, necessary ions and molecules, and a suitable temperature and pH). In some embodiments, the adapters each comprise a primer binding site. As used herein, a “primer binding site” or “primer binding sequence” refers to a sequence for facilitating the binding of a primer. For example, in some implementations, the primer binding sequence provides a site that is reverse complementary to a sequence in a PCR primer.

In some embodiments, the adapters each comprise an index sequence. As used herein, an “index sequence” (also known as a tag sequence) refers to a polynucleotide sequence that is added to each nucleic sequence fragment during library preparation and can be associated with one or more nucleic acid molecules.

In some embodiments, the adapters each comprise a barcode. A “barcode” may refer to a sample barcode, spatial barcode or single cell sequencing barcode. A “barcode” may refer to a unique nucleotide sequence ligated to fragments within a sequencing library for downstream in silico sorting and identification. As used herein, a “sample barcode” refers to a short nucleotide tag added to sequences of interest during sample preparation to provide information about the cell, cell type, or other feature of the sample for each sequence. As used herein, a “spatial barcode” or “spatial sequencing barcode” is a unique nucleotide identifier that allows the location of a gene transcript to be mapped within a tissue sample. Spatial barcodes are commonly based on a barcoding chemistry strategy that uses chemical reactions to allocate unique identifiers to target molecules as barcodes for precise spatial and genomic-scale transcript quantitative analyses. As used herein, a “single cell sequencing barcode” refers to a barcode added to sequences of interest from isolated single cells, allowing separation cell by cell.

In some embodiments, the adapter is substantially non-complementary to the 3′ end or the 5′ end of any source nucleic acid sequence present in the sample. Generally, the adapter can include any combination of nucleotides and/or nucleic acids. In some aspects, the adapter can include one or more cleavable groups at one or more locations. In another aspect, the adapter can include a sequence that is substantially identical, or substantially complementary, to at least a portion of a primer, for example a universal primer. In some embodiments, the adapter can include an index sequence (also referred to as a tag) to assist with downstream error correction, identification or sequencing. The terms “adapter” and “adaptor” are used interchangeably.

Described herein are a pool of adapters, wherein the adapters each comprise a unique molecular identifier (UMI), wherein each UMI comprises an oligonucleotide sequence comprising the sequence 5′-X_{N}YZ-3′, wherein the 3′ end of the UMI ends with YZ, wherein YZ does not correspond to GA, GC, or GG, wherein N is an integer greater than or equal to 1 and each X is any nucleic acid and the same or different, and wherein each adapter is attached to a solid support.

Also described herein are a pool of adapters, wherein the adapters each comprise a primer, a primer binding site, an index sequence, and/or a barcode, wherein each adapter comprises an oligonucleotide sequence comprising the sequence 5′-X_{N}YZ-3′, wherein the 3′ end of the adapter ends with YZ, wherein YZ does not correspond to GA, GC, or GG, wherein N is an integer greater than or equal to 1 and each X is the same or different, and wherein in each adapter is attached to a solid support.

In some embodiments, each N is an integer from 1 to 40, 1 to 30, 1 to 20, or 1 to 10. In some embodiments, each N is an integer from 5-20. In some embodiments, each N is an integer from 5-15. In some embodiments, each N is an integer from 5-10. In some embodiments, each N is 2, 3, 4, 5, 6, 7, 8, 9, 10, 11, 12, 13, 14, 15, 16, 17, 18, 19, 20, 21, 22, 23, 24, 25, 26, 27, 28, 29 30, 31, 32, 33, 34, 35, 36, 37, 38, 39, or 40.

In some embodiments, each N is an integer from 2 to 7. In some embodiments, each N is an integer from 4 to 7. In some embodiments, each N is an integer from 4 to 5. In some embodiments, each N is an integer from 4 to 6. In some embodiments, each N is an integer from 5 to 6. In some embodiments, each N is an integer from 5 to 7. In some embodiments, wherein each N is an integer from 6 to 7. In some embodiments, the UMIs have the same length and in other embodiments the UMIs have different lengths.

In some embodiments, YZ corresponds to TX, wherein X is any nucleic acid. In some embodiments, YZ corresponds to TA, TC or TG.

In some embodiments, the adapters comprise DNA. In some embodiments, the adapters comprise RNA. In some embodiments, the adapters comprise an RNA: DNA hybrid. In some embodiments, the adapters are methylated.

In some embodiments, the adapters are single stranded.

In some embodiments, the adapters are double stranded. In some embodiments, the double stranded adapters each comprise a UMI, wherein the UMI is on only one strand. In some embodiments, the double stranded adapters each comprise a UMI, wherein the UMI is on both strands.

In some embodiments, each adapter in the pool of adapters comprises a UMI and wherein the UMI is a unique UMI shared by no other adapter in the pool of adapters.

In some embodiments, each adapter in the pool of adapters comprises a UMI and more than one adapter in the pool of adapters has the same UMI, but wherein that UMI differs from other adapters in the pool of adapters. A person of skill in the art can optimize the number of UMIs needed based on sample input and desired sequencing depth.

A wide variety of solid supports may be used to immobilize oligonucleotides for sequencing as described herein, including those described in WO 2014/108810. In some embodiments, the solid support is a flowcell. In some embodiments, the solid support is a bead.

### C. Nucleic Acids

As used herein, the term “nucleic acid” is intended to be consistent with its use in the art and includes naturally occurring nucleic acids or functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g., found in deoxyribonucleic acid (DNA)) or a ribose sugar (e.g., found in ribonucleic acid (RNA)). A nucleic acid can contain any of a variety of analogs of these sugar moieties that are known in the art. A nucleic acid can include native or non-native bases. In this regard, a native deoxyribonucleic acid can have one or more bases chosen from adenine, thymine, cytosine or guanine and a ribonucleic acid can have one or more bases chosen from uracil, adenine, cytosine, or guanine. Useful non-native bases that can be included in a nucleic acid are known in the art. The term “target,” when used in reference to a nucleic acid, is intended as a semantic identifier for the nucleic acid in the context of a method or composition set forth herein and does not necessarily limit the structure or function of the nucleic acid beyond what is otherwise explicitly indicated.

The term “read” refers to a sequence read from a portion of a nucleic acid sample. Typically, though not necessarily, a read represents a short sequence of contiguous base pairs in the sample. The read may be represented symbolically by the base pair sequence in A. T. C, and G of the sample portion, together with a probabilistic estimate of the correctness of the base (quality score). It may be stored in a memory device and processed as appropriate to determine whether it matches a reference sequence or meets other criteria. A read may be obtained directly from a sequencing apparatus or indirectly from stored sequence information concerning the sample. In some cases, a read is a DNA sequence of sufficient length (e.g., at least about 20 bp) that can be used to identify a larger sequence or region, e.g., that can be aligned and mapped to a chromosome or genomic region or gene.

As used herein, the term “reference genome” or “reference sequence” refers to any particular known genetic sequence, whether partial or complete, of any organism or virus which may be used to reference identified sequences from a subject. For example, a reference genome used for human subjects as well as many other organisms is found at the National Center for Biotechnology Information at ncbi.nlm nih.gov. A “genome” refers to the complete genetic information of an organism or virus, expressed in nucleic acid sequences. However, it is understood that “complete” is a relative concept, because even the gold-standard reference genome is expected to include gaps and errors.

The term “primer,” as used herein refers to an isolated oligonucleotide that is capable of acting as a point of initiation of synthesis when placed under conditions inductive to synthesis of an extension product (e.g., the conditions include nucleotides, an inducing agent such as DNA polymerase, necessary ions and molecules, and a suitable temperature and pH). The primer may be preferably single stranded for maximum efficiency in amplification, but alternatively may be double stranded. If double stranded, the primer is first treated to separate its strands before being used to prepare extension products. The primer may be an oligodeoxyribonucleotide (in other words, an oligo comprised of DNA), but it may also be comprised of other nucleic acids. The primer is sufficiently long to prime the synthesis of extension products in the presence of the inducing agent. The exact lengths of the primers will depend on many factors, including temperature, source of primer, use of the method, and the parameters used for primer design. In some embodiments, the primer may comprise i5 or i7 index sequences. One embodiment of this is shown in FIG. 1B, where indexes are added during PCR after adapter ligation.

The terms “P5” and “P7” may be used when referring to amplification primers, e.g., universal primer extension primers. The terms “P5” (P5 prime) and “P7” (P7 prime) refer to the complement of P5 and P7, respectively. It will be understood that any suitable amplification primers can be used in the methods presented herein, and that the use of P5 and P7 are exemplary embodiments only.

Uses of amplification primers such as P5 and P7 on flow cells is known in the art, as exemplified by the disclosures of WO 2007/010251, WO 2006/064199, WO 2005/065814, WO 2015/106941, WO 1998/044151, and WO 2000/018957. For example, any suitable forward amplification primer, whether immobilized or in solution, can be useful in the methods presented herein for hybridization to a complementary sequence and amplification of a sequence. Similarly, any suitable reverse amplification primer, whether immobilized or in solution, can be useful in the methods presented herein for hybridization to a complementary sequence and amplification of a sequence. One of skill in the art will understand how to design and use primer sequences that are suitable for capture, and amplification of nucleic acids as presented herein.

The terms “upstream” and “5′-of” with reference to positions in a nucleic acid sequence are used interchangeably to refer to a relative position in the nucleic acid sequence that is further towards the 5′ end of the sequence.

The terms “downstream” and “3′-of” with reference to positions in a nucleic acid sequence are used interchangeably to refer to a relative position in the nucleic acid sequence that is further towards the 3′ end of the sequence.

### II. Methods of Use

### A. Methods of Sequencing Using UMIs

The disclosure provides methods for sequencing source nucleic acid from a sample using any of the pool of adapters disclosed herein in the discussion of Compositions in Section I above. In some embodiments, the adapters each comprise a unique molecular identifier (UMI), wherein each UMI comprises an oligonucleotide sequence comprising the sequence 5′-X_{N}YZ-3′, wherein the 3′ end of the UMI ends with YZ, wherein YZ does not correspond to GA, GC, or GG, wherein N is an integer greater than or equal to 1 and each X is the same or different, wherein each adapter is attached to a solid support, and wherein each UMI is an oligonucleotide sequence that can be used to identify an individual molecule of a source nucleic acid fragment in the sample, comprising: (a) fragmenting source nucleic acid molecules into nucleic acid fragments; (b) applying the pool of adapters to source nucleic acid fragments in the sample; (c) amplifying both strands of the source nucleic acid-adapter products to obtain a plurality of amplified polynucleotides: (d) sequencing the plurality of amplified polynucleotides, thereby obtaining a plurality of reads each associated with a UMI; (e) identifying a plurality of UMIs associated with the plurality of reads: (f) determining sequences of the source nucleic acid fragments in the sample using the plurality of reads obtained in (c) and the plurality of UMIs identified in (d).

This disclosure also provides methods for sequencing source nucleic acid from a sample using any of the pool of adapters disclosed herein in the discussion of Compositions in Section I above. In some embodiments, the adapters each comprise a primer, a primer binding site, an index sequence, and/or a barcode, wherein each adapter comprises an oligonucleotide sequence comprising the sequence 5′-X_{N}YZ-3′, wherein the 3′ end of the adapter ends with YZ, wherein YZ does not correspond to GA, GC, or GG, wherein N is an integer greater than or equal to 1 and each X is the same or different, and wherein each adapter is attached to a solid support, comprising: (a) fragmenting source nucleic acid molecules into nucleic acid fragments; (b) applying the pool of adapters to source nucleic acid fragments in the sample; (c) amplifying both strands of the source nucleic acid-adapter products to obtain a plurality of amplified polynucleotides; and (d) sequencing the plurality of amplified polynucleotides.

A wide variety of library preparation methods comprising a step of adapter ligation are known in the art, such as TruSeq and TruSight Oncology 500 (See, e.g., TruSeq®) RNA Sample Preparation v2 Guide, 15026495 Rev. F, Illumina, 2014). Exemplary ligated forked adapters are discussed in WO 2007/052006, US Patent Pub. No. 2020/0080145, U.S. Pat. No. 9,868,982, and WO 2020/144373. In particular, adapter ligation may allow for more flexible incorporation of adapters (such as adapters with longer lengths) as compared to methods of tagging fragments via tagmentation (wherein adapter sequences are incorporated into fragments during the transposition reaction). In some methods involving tagmentation, additional adapter sequences may be incorporated by PCR reactions, and the present methods may obviate the need for an additional PCR step to incorporate additional adapter sequences.

Ligation technology is commonly used to prepare NGS libraries for sequencing. In some embodiments, the ligation step uses an enzyme to connect adapters to one or both ends of nucleic acid fragments. In some embodiments, an A-base is added to blunt ends of each strand, preparing them for ligation to the sequencing adapters. In some embodiments, each adapter contains a T-base overhang, providing a complementary overhang for ligating the adapter to the A-tailed fragmented DNA.

Adapter ligation protocols are known to have advantages over other methods. For example, adapter ligation can be used to generate the full complement of sequencing primer hybridization sites for single, paired-end, and indexed reads. In some embodiments, adapter ligation eliminates a need for additional PCR steps to add an index tag and index primer sites.

In some embodiments, the adapters each comprise a unique molecular identifier (UMI), wherein a first UMI is on the first strand of the double-stranded source nucleic acid fragments.

In some embodiments, the adapters each comprise a unique molecular identifier (UMI), wherein a first copy of the first UMI is on the first strand and a second copy of the first UMI is on the second strand of the double-stranded source nucleic acid fragments.

In some embodiments, the adapters each comprise a unique molecular identifier (UMI), wherein a first UMI is on a first strand of the double-stranded source nucleic acid fragments, a second UMI is on the second strand of the double-stranded target nucleic acid fragments.

Also disclosed herein are methods of sequencing a double-stranded nucleic acid library produced by any of the methods described herein, wherein the adapters each comprise a unique molecular identifier (UMI), wherein the UMIs are sequenced to provide increased sensitivity in DNA sequencing.

### B. Samples

A biological sample used in accordance with the present disclosure can be any type that comprises source nucleic acids (i.e., target nucleic acids). However, the sample need not be completely purified, and can comprise, for example, nucleic acid mixed with protein, other nucleic acid species, other cellular components, and/or any other contaminant. In some embodiments, the biological sample comprises a mixture of nucleic acid, protein, other nucleic acid species, other cellular components, and/or any other contaminant present in approximately the same proportion as found in vivo. For example, in some embodiments, the components are found in the same proportion as found in an intact cell. In some embodiments, the sample may be from a mammal. In some embodiments the sample may be from a human, monkey, rat and/or mouse.

In some embodiments, samples may be from a patient. In some embodiments, samples may be from a patient with cancer (i.e., an oncology sample). In some embodiments, samples may be from a patient with a rare disease. In some embodiments, samples may be from a patient with a viral infection. In some embodiments, the sample may be a tumor sample. In some embodiments, the sample may be a blood sample. In some embodiments the sample may be a tissue sample.

In some embodiments, samples may be derived from a biological fluid, cell, tissue, organ, or organism, that includes a nucleic acid or a mixture of nucleic acids having at least one nucleic acid sequence that is to be screened for copy number variation and other genetic alterations, such as, but not limited to, single nucleotide polymorphism, insertions, deletions, and structural variations. In certain embodiments the sample has at least one nucleic acid sequence whose copy number is suspected of having undergone variation. Such samples include, but are not limited to sputum/oral fluid, amniotic fluid, blood, a blood fraction, or fine needle biopsy samples, urine, peritoneal fluid, pleural fluid, and the like. Although the sample is often taken from a human subject (e.g., a patient), the assays can be used for samples from any mammal, including, but not limited to dogs, cats, horses, goats, sheep, cattle, pigs, etc., as well as mixed populations, as microbial populations from the wild, or viral populations from patients.

In some embodiments, samples may be used directly as obtained from the biological source or following a pretreatment to modify the character of the sample. Some pretreatment do not impact the nucleic acids in the sample, whereas other pretreatments do impact the nucleic acids in the sample. For example, such pretreatment may include preparing plasma from blood, diluting viscous fluids, and so forth. Methods of pretreatment may also involve, but are not limited to, filtration, precipitation, dilution, distillation, mixing, centrifugation, freezing, lyophilization, concentration, nucleic acid fragmentation, inactivation of interfering components, the addition of reagents, lysing, etc. If such methods of pretreatment are employed with respect to the sample, such pretreatment methods are typically such that the nucleic acid(s) of interest remain in the test sample, sometimes at a concentration proportional to that in an untreated test sample (e.g., namely, a sample that is not subjected to any such pretreatment method(s)). Such “treated” or “processed” samples are still considered to be biological “test” samples with respect to the methods described herein.

In some embodiments, the source nucleic acids are double-stranded. In some embodiments the source nucleic acids are double-stranded DNA. In some embodiments, the double-stranded source nucleic acids are ctDNA. In some embodiments, the double-stranded source nucleic acids are cfDNA. In some embodiments, the double-stranded source nucleic acids are RNA.

In some embodiments, the sample comprises a source double-stranded DNA. In some embodiments, the DNA is genomic DNA. In some embodiments, the DNA is cell-free DNA (cfDNA). In some embodiments, the DNA is circulating tumor DNA (ctDNA).

In some embodiments, the source nucleic acids are single-stranded. In some embodiments, the sample comprises source RNA. In some embodiments, the sample comprises RNA and DNA. In some embodiments, the source RNA is mRNA.

In some embodiments, the source RNA is messenger RNA (mRNA), transfer RNA (tRNA), or ribosomal RNA (rRNA). Appropriate capture oligonucleotides could be designed based on the type of source RNA.

In some embodiments, the source RNA is mRNA. In some embodiments, the source RNA is polyadenylated (i.e., comprises a stretch of RNA that contains only adenine bases). In some embodiments, the mRNA comprises polyA tails. In some embodiments, the 3′ ends of the mRNA comprise poly A tails.

In some embodiments, cDNA synthesis is performed by a reverse transcriptase. In some embodiments, this cDNA synthesis yield DNA: RNA duplexes, wherein a strand of DNA is generated that can hybridize to a strand of RNA. In some embodiments, a reverse transcriptase polymerase is added to a sample comprising RNA under conditions to synthesize cDNA. In some embodiments, conditions to synthesize cDNA include the presence of nucleotides and/or primers that can bind to RNA (such as polyT primers and/or randomer primers).

### C. Library Preparation

As used herein, the term “library” refers to a collection of members. In one embodiment, the library includes a collection of nucleic acid members, for example, a collection of whole genomic, subgenomic fragments, cDNA, cDNA fragments, RNA, RNA fragments, or a combination thereof. In some embodiments, a portion or all library members include an amplification adapter sequence. The amplification adapter sequence can be located at one or both ends. The amplification adapter sequence can be used in, for example, a sequencing method (for example, an NGS method), for amplification, for reverse transcription, or for cloning into a vector.

In some embodiments, this DNA: RNA hybrid-specific cleavage comprises use of RNase H. This methodology is implemented as part of the current Illumina Total RNA Stranded Library Prep workflow and New England Biolabs NEBNext rRNA Depletion Kit and RNA depletion methods as described in U.S. Pat. Nos. 9,745,570 and 9,005,891.

### D. Amplification

In some embodiments, methods described herein comprise one or more amplification steps. In some embodiments, library fragments are amplified before being added to a solid support. In some embodiments library fragments are amplified after a method of depleting or enriching. In some embodiments, amplifying is by PCR amplification.

As used herein, “amplify,” “amplifying.” or “amplification reaction” and their derivatives, refer generally to any action or process whereby at least a portion of a nucleic acid molecule is replicated or copied into at least one additional nucleic acid molecule. The additional nucleic acid molecule optionally includes a sequence that is substantially identical or substantially complementary to at least some portion of the template nucleic acid molecule. The template nucleic acid molecule can be single-stranded or double-stranded and the additional nucleic acid molecule can independently be single-stranded or double-stranded. Amplification optionally includes linear or exponential replication of a nucleic acid molecule. In some embodiments, such amplification can be performed using isothermal conditions: in other embodiments, such amplification can include thermocycling. In some embodiments, the amplification is a multiplex amplification that includes the simultaneous amplification of a plurality of target sequences in a single amplification reaction. In some embodiments, “amplification” includes amplification of at least some portion of DNA and RNA based nucleic acids alone, or in combination. The amplification reaction can include any of the amplification processes known to one of ordinary skill in the art. In some embodiments, the amplification reaction includes polymerase chain reaction (PCR).

In some embodiments, collected library fragments are amplified.

In some embodiments, the amplifying is performed with a thermocycler. In some embodiments, the amplifying is by PCR amplification.

As used herein, the term “polymerase chain reaction” (“PCR”) refers to the method as described in U.S. Pat. Nos. 4,683,195 and 4,683,202, which describe a method for increasing the concentration of a segment of a polynucleotide of interest in a mixture of genomic DNA without cloning or purification. This process for amplifying the polynucleotide of interest comprises introducing a large excess of two oligonucleotide primers to the DNA mixture containing the desired polynucleotide of interest, followed by a series of thermal cycling in the presence of a DNA polymerase. The two primers are complementary to their respective strands of the double stranded polynucleotide of interest. The mixture is denatured at a higher temperature first and the primers are then annealed to complementary sequences within the polynucleotide of interest molecule. Following annealing, the primers are extended with a polymerase to form a new pair of complementary strands. The steps of denaturation, primer annealing, and polymerase extension can be repeated many times (referred to as thermocycling) to obtain a high concentration of an amplified segment of the desired polynucleotide of interest. The length of the amplified segment of the desired polynucleotide of interest (amplicon) is determined by the relative positions of the primers with respect to each other, and therefore, this length is a controllable parameter. By virtue of repeating the process, the method is referred to as the “polymerase chain reaction” (hereinafter “PCR”). Because the desired amplified segments of the polynucleotide of interest become the predominant nucleic acid sequences (in terms of concentration) in the mixture, they are said to be “PCR amplified.” In a modification to the method discussed above, the source nucleic acid molecules can be PCR amplified using a plurality of different primer pairs, in some cases, one or more primer pairs per target nucleic acid molecule of interest, thereby forming a multiplex PCR reaction.

In some embodiments, the amplifying is performed without PCR amplification. In some embodiments, the amplifying does not require a thermocycler.

In some embodiments, the amplifying is performed without a thermocycler. In some embodiments, the amplifying is performed by bridge or cluster amplification.

### E. Sequencing of Libraries

In some embodiments, a library produced according to the methods provided herein is sequenced.

Libraries can be sequenced according to any suitable sequencing methodology, such as direct sequencing, including sequencing by synthesis (SBS), sequencing by ligation, sequencing by hybridization, nanopore sequencing and the like. In some embodiments, the libraries are sequenced on a solid support. In some embodiments, the solid support for sequencing is the same solid support upon which amplification occurs.

Flow cells provide a convenient solid support for performing sequencing. The term “flow cell” as used herein refers to a chamber comprising a solid surface across which one or more fluid reagents can be flowed. Examples of flow cells and related fluidic systems and detection platforms that can be readily used in the methods of the present disclosure are described, for example, in Bentley et al., Nature 456:53-59 (2008); WO 04/018497; WO 91/06678; WO 07/123744; U.S. Pat. Nos. 7,057,026; 7,211,414; 7,315,019; 7,329,492; 7,405,281; and US Pat. Publication No. 2008/0108082. While standard flowcells used for imaging may be employed in the present methods, flowcells can also be engineered differently than flowcells intended for imaging. In some embodiments, a flowcell may have a high density of immobilized oligonucleotides, wherein imaging infrastructure would have difficulty separating out into different bridge-amplified clusters associated with different immobilized oligonucleotides. In some embodiments, a high density of immobilized oligonucleotides improves hybridization efficiency. In some embodiments, standard clear glass may be used in a flowcell. In other embodiments, hard plastic may be used in the flowcell. Use of glass in a flowcell may allow use of a standard flowcell without further optimization, whereas use of hard plastic may reduce the cost of manufacturing the flowcell and/or improve stability of a flowcell. Depending on the advantages desired, different materials may be used. In some embodiments, immobilized oligonucleotides are embedded in a substrate other than that of a standard flowcell (i.e., embedded in a substrate other than PAZAM) to improve immobilization of oligonucleotides of longer length.

One or more library fragments (or amplicons produced from library fragments) in such a format can be subjected to an SBS or other detection technique that involves repeated delivery of reagents in cycles. In SBS, extension of a nucleic acid primer along a nucleic acid template (e.g., a target nucleic acid or amplicon thereof) is monitored to determine the sequence of nucleotides in the template. The underlying chemical process can be polymerization (e.g., as catalyzed by a polymerase enzyme). In a particular polymerase-based SBS embodiment, fluorescently labeled nucleotides are added to a primer (thereby extending the primer) in a template dependent fashion such that detection of the order and type of nucleotides added to the primer can be used to determine the sequence of the template. Exemplary SBS procedures, fluidic systems and detection platforms that can be readily adapted for use with amplicons produced by the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082.

Other sequencing procedures that use cyclic reactions can be used, such as pyrosequencing. Pyrosequencing detects the release of inorganic pyrophosphate (pPi) as particular nucleotides are incorporated into a nascent nucleic acid strand (Ronaghi, et al., Analytical Biochemistry 242 (1), 84-9 (1996); Ronaghi, Genome Res. 11 (1), 3-11 (2001); Ronaghi et al. Science 281 (5375), 363 (1998); U.S. Pat. Nos. 6,210,891; 6,258,568 and 6,274,320. In pyrosequencing, released pPi can be detected by being immediately converted to adenosine triphosphate (ATP) by ATP sulfurylase, and the level of ATP generated can be detected via luciferase-produced photons. Thus, the sequencing reaction can be monitored via a luminescence detection system. Excitation radiation sources used for fluorescence-based detection systems are not necessary for pyrosequencing procedures. Useful fluidic systems, detectors and procedures that can be adapted for application of pyrosequencing to amplicons produced according to the present disclosure are described, e.g., in US 2016/0199513 A1, US 2005/0191698 A1, U.S. Pat. Nos. 7,595,883, and 7,244,559.

Some embodiments can utilize methods involving the real-time monitoring of DNA polymerase activity. For example, nucleotide incorporations can be detected through fluorescence resonance energy transfer (FRET) interactions between a fluorophore-bearing polymerase and γ-phosphate-labeled nucleotides, or with zeromode waveguides (ZMWs). Techniques and reagents for FRET-based sequencing are described, e.g., in Levene et al. Science 299, 682-686 (2003); Lundquist et al. Opt. Lett. 33, 1026-1028 (2008); Korlach et al. Proc. Natl. Acad. Sci. USA 105, 1176-1181 (2008).

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1. Methods set forth herein for amplifying target nucleic acids using kinetic exclusion can be readily applied to substrates used for detecting protons. More specifically, methods set forth herein can be used to produce clonal populations of amplicons that are used to detect protons.

Another useful sequencing technique is nanopore sequencing (see, e.g., Deamer et al. Trends Biotechnol. 18, 147-151 (2000); Deamer et al. Acc. Chem. Res. 35:817-825 (2002); Li et al. Nat. Mater. 2:611-615 (2003). In some nanopore embodiments, the target nucleic acid or individual nucleotides removed from a target nucleic acid pass through a nanopore. As the nucleic acid or nucleotide passes through the nanopore, each nucleotide type can be identified by measuring fluctuations in the electrical conductance of the pore. (U.S. Pat. No. 7,001,792; Soni et al. Clin. Chem. 53, 1996-2001 (2007); Healy, Nanomed. 2, 459-481 (2007); Cockroft et al. J. Am. Chem. Soc. 130, 818-820 (2008).

Exemplary methods for array-based expression and genotyping analysis that can be applied to detection according to the present disclosure are described in U.S. Pat. Nos. 7,582,420; 6,890,741; 6,913,884 or 6,355,431 or US Patent Pub. Nos. 2005/0053980 A1; 2009/0186349 A1 or US 2005/0181440 A1.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more nucleic acid fragments, the system comprising components such as pumps, valves, reservoirs, fluidic lines and the like. A flow cell can be configured and/or used in an integrated system for detection of target nucleic acids. Exemplary flow cells are described, e.g., in US 2010/0111768 A1 and US 2012/0270305 A1. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods.

In some embodiments, a method of sequencing a UMI library of the present disclosure comprises sequencing the UMIs to provide increased sensitivity in DNA sequencing.

Throughout this application and claims, the term “and/or” means one or more of the listed elements or a combination of any two or more of the listed elements.

The term “comprises” and variations thereof do not have a limiting meaning where these terms appear in the description and claims. The terms “includes”, “including”, “has”, or “having”, and all variations thereof mean the same thing as “comprises.”

Unless otherwise specified, “a,” “an,” “the,” and “at least one” are used interchangeably and mean one or more than one.

As used herein, the term “each,” when used in reference to a collection of items, is intended to identify an individual term in the collection but does not necessarily refer to every term in the collection unless the context clearly dictates otherwise.

The recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Unless defined otherwise, all technical and scientific terms used herein have the same meaning as commonly understood by one of ordinary skill in the art pertinent to the methods and compositions described. All patents, applications, published applications and other publications referred to herein are incorporated by reference in their entirety. If a definition set forth in this section is contrary to or otherwise inconsistent with a definition set forth in the patents, applications, published applications, and other publications that are herein incorporated by reference, the definition set forth in this section prevails over the definition that is incorporated herein by reference.

All headings are for the convenience of the reader and should not be used to limit the meaning of the text that follows the heading, unless so specified.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

### EXAMPLES

### Example 1. Performance and Functional Testing of UMIs

Analysis of 120 established 6- or 7mer UMIs indicated that a subgroup of adapters was more present after adapter ligation in a standard library preparation protocol than others (FIG. 1A). Thus, this subgroup was tested against 120 established UMI adapters (120 UMI) to evaluate if certain UMIs led to better assay performance.

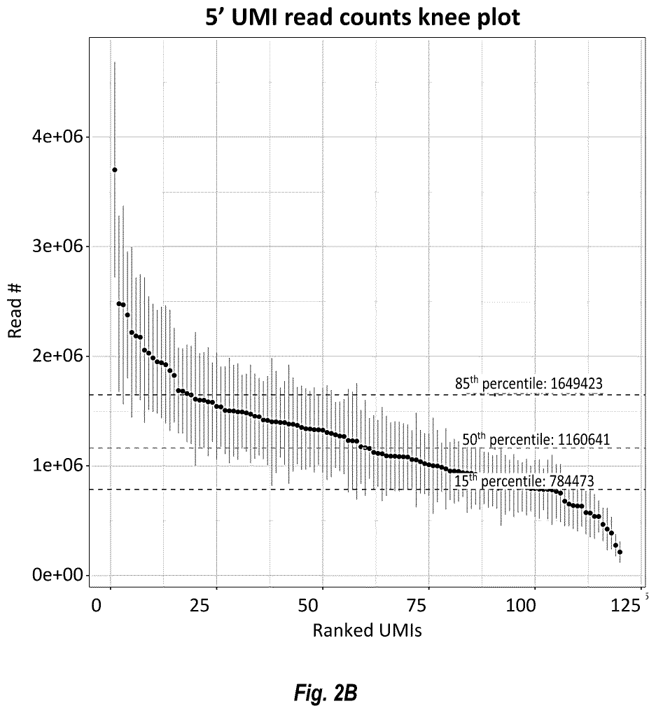

To evaluate performance, the UMIs were evaluated for ligation efficiency using 720 samples comprising cfDNA extracted from the blood of human donors, cell lines, and synthetic cfDNA samples. Cell lines used had known markers-used to test marker detection. Synthetic cfDNA was mixed to mimic a specific set of markers and used as controls. The UMIs were ranked by average # of reads assigned to the UMI both 3′ and 5′ ends of the fragment (FIGS. 2A-2B).

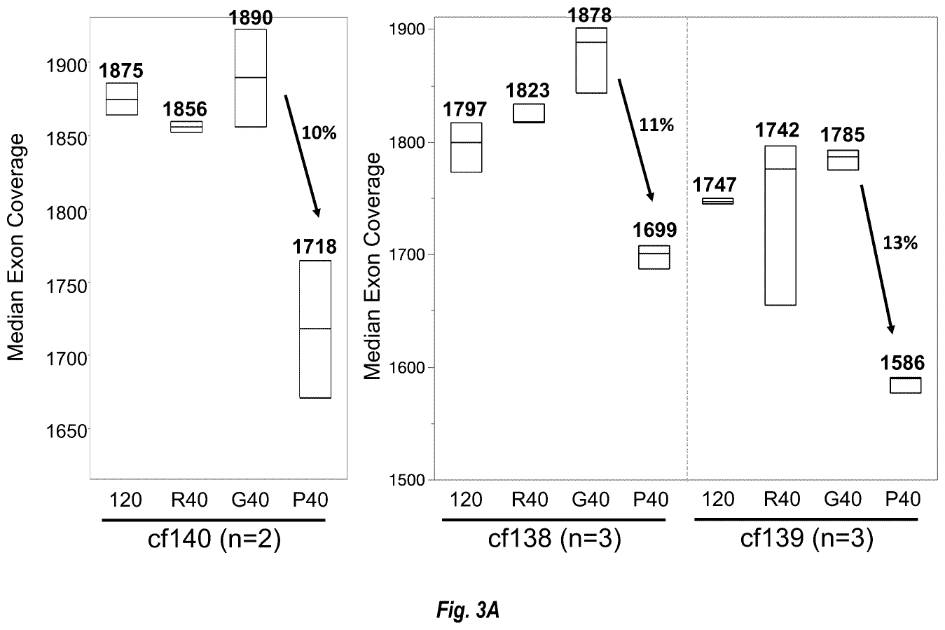

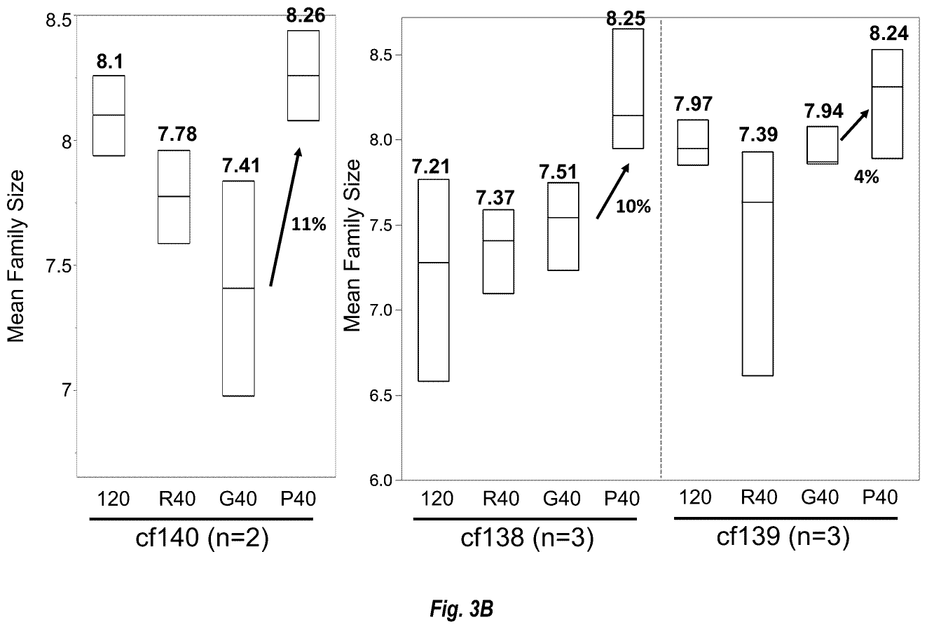

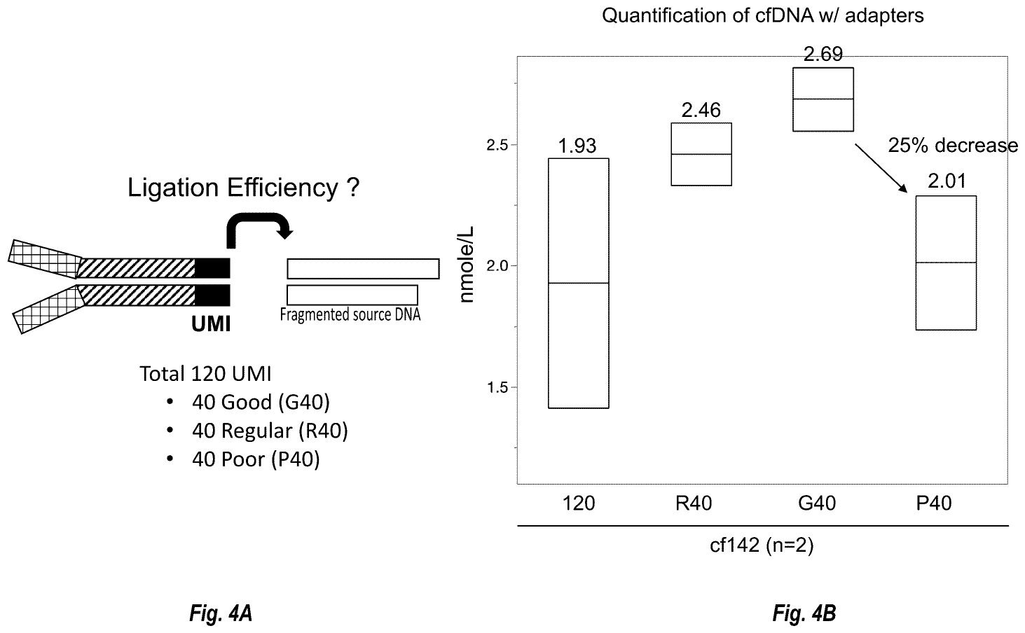

The ranked UMIs were separated into 40 Regular (R40), 40 Good (G40), and 40 Poor (P40) performing UMIs, as shown in FIG. 4A. The G40 UMIs performed with 10-13% better exon coverage than P40 UMIs (FIG. 3A), and similarly performed better than P40 with 4-11% mean family size (FIG. 3B). FIG. 4B shows that the P40 UMIs were associated with a 25% lower amount of cfDNA with adapters indicating that the ligation was less efficient.

Based on these results it was hypothesized that an extension could be added to the 3′ end of UMIs (closer to where ligation takes place) to normalize and maximize ligation efficiency.

### Example 2. UMI Sequence Analysis

Sequences of the 40 good and 40 poor UMIs were analyzed. A summary of these analyses is present in Tables 1 and 2 below.

TABLE 1
Sequence of Ends of UMIs

| UMI 3′ end | G40 | P40 |
|---|---|---|
| TA | 10.0% | 2.5% |
| TC | 17.5% | 2.5% |
| TG | 35.0% | 0.0% |
| GA | 0.0% | 17.5% |
| GC | 0.0% | 27.5% |
| GG | 0.0% | 0.0% |

TABLE 2
Identity of Nucleotide at the Two Most 3′
Positions of the 6- or 7-mer Oligonucleotide

| 6-/7-mer | Good UMI | Poor UMI |
|---|---|---|
| Pos. 5/6 | T | G |

| | |
|---|---|
| Pos. 6/7 | A/G/C |

The percentage of UMIs in each population that end in TA/TC/TG (associated with good performance), and GA/GC/GG (associated with poor performance) are shown in Table 1. These data indicated that the second position from the 3′ end of the UMI is enriched in T in Good UMIs and in G in Poor UMIs for both 6- and 7-mers, with the last position appearing variable. As summarized in Table 2, a T in position 5 or 6 (in a 6- or 7-mer, respectively) of the UMI was associated with improved performance. Conversely, a G in the same position was associated with poor performance.

### Example 3. Functional Testing of Improved UMIs

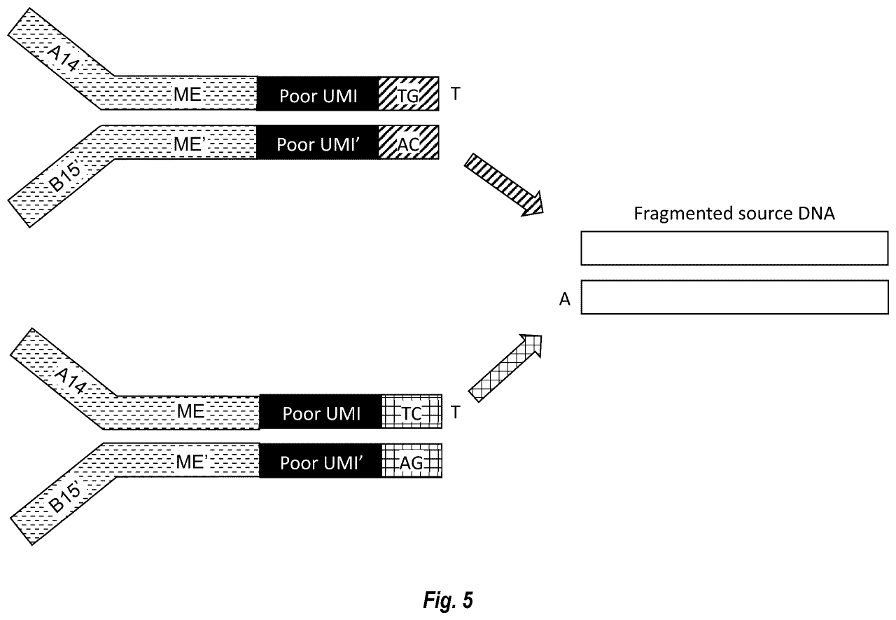

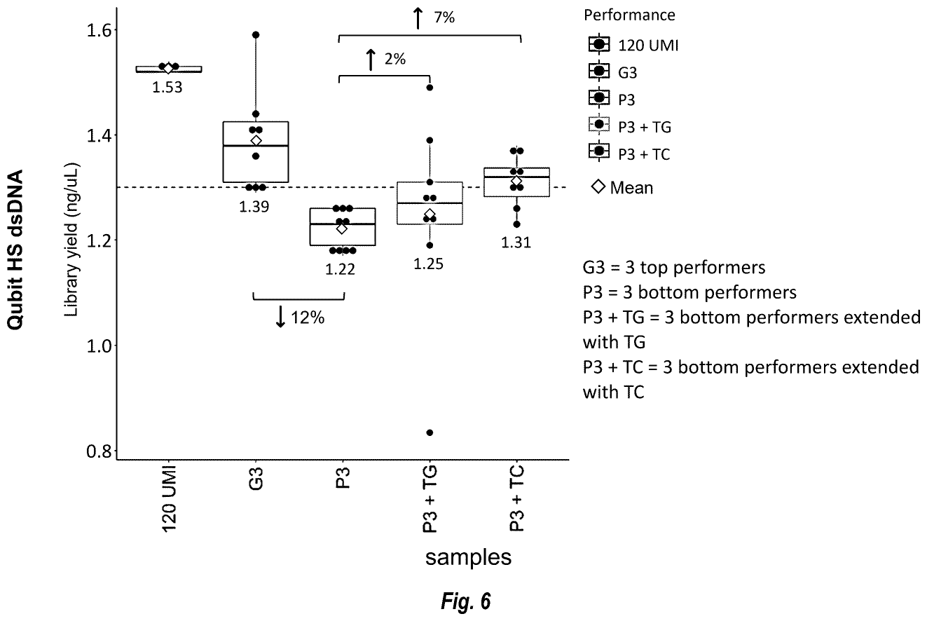

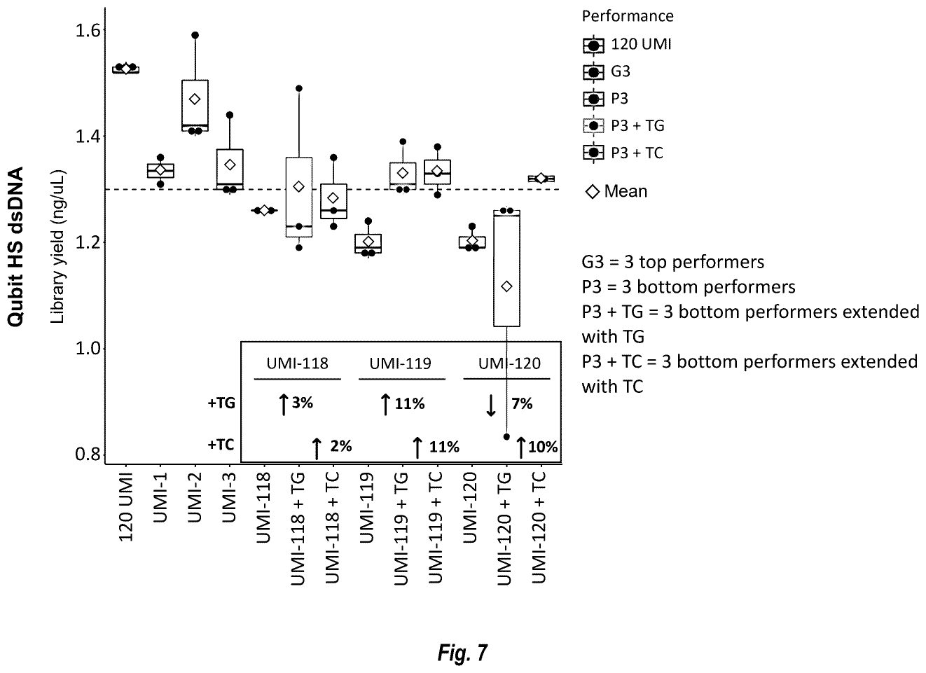

To test whether UMI performance could be improved by the addition of an extension at the 3′ end of UMI, the three worst performing UMIs (UMI-118, UMI-119, and UMI-120) were extended with TG or TC as shown in FIG. 5. The library yield was measured comparing the pool of 120 UMIs (120 UMI), the top 3 performing UMIs (G3), the 3 worst performing UMIs (P3) and the 3 worst performing UMIs extended with TG (P3+TG) or TC (P3+TC). The results of the library yield as measured using a Qubit™ dsDNA Assay are shown in FIG. 6. FIG. 7 shows the data of the results in FIG. 6, for each individual UMI. TG- and TC-extension of poor performing UMIs resulted in 2-11% yield increase compared with the original sequences. Specifically, UMI-118 had a 3% increase in library yield with a -TG extension, and 2% increase in library yield with a TC extension, UMI-119 had a 11% increase in library yield with a -TG extension, and 11% increase in library yield with a TC extension, and UMI-120 had a 7% decrease in library yield with a -TG extension, and 10% increase in library yield with a TC extension.

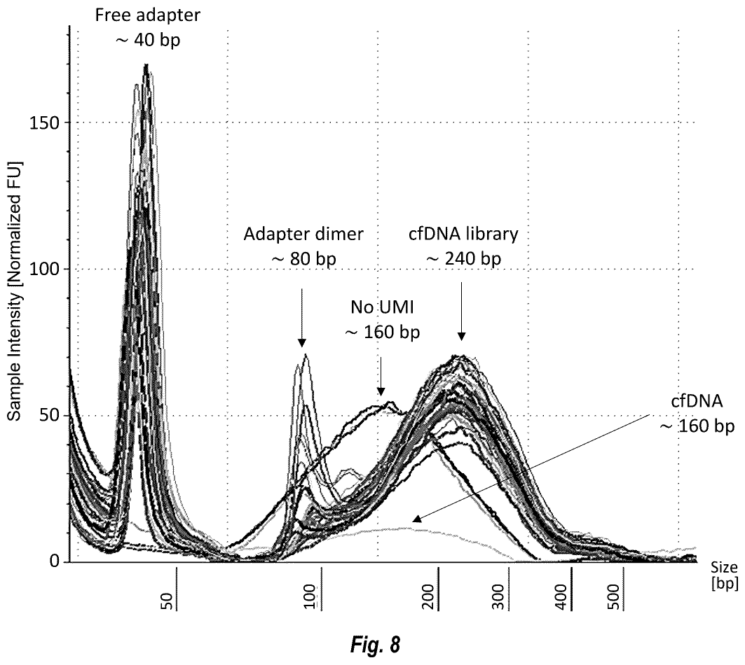

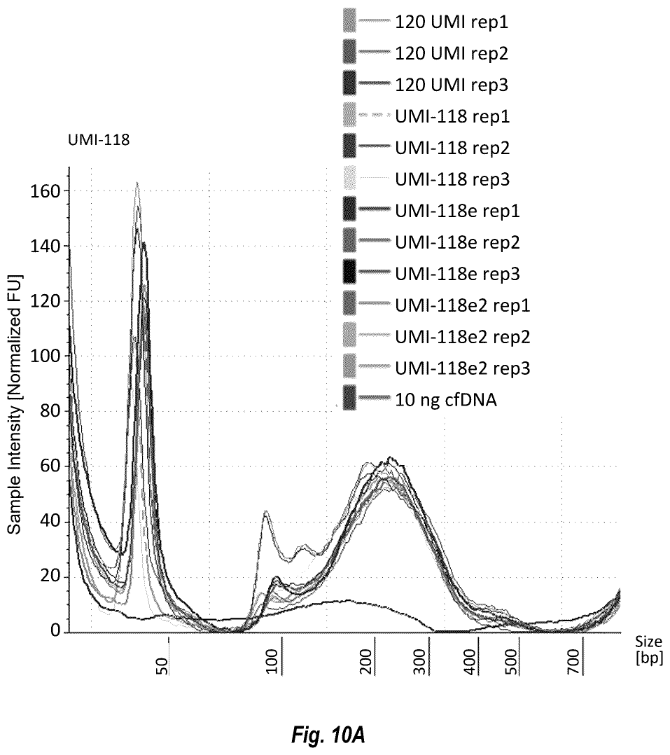

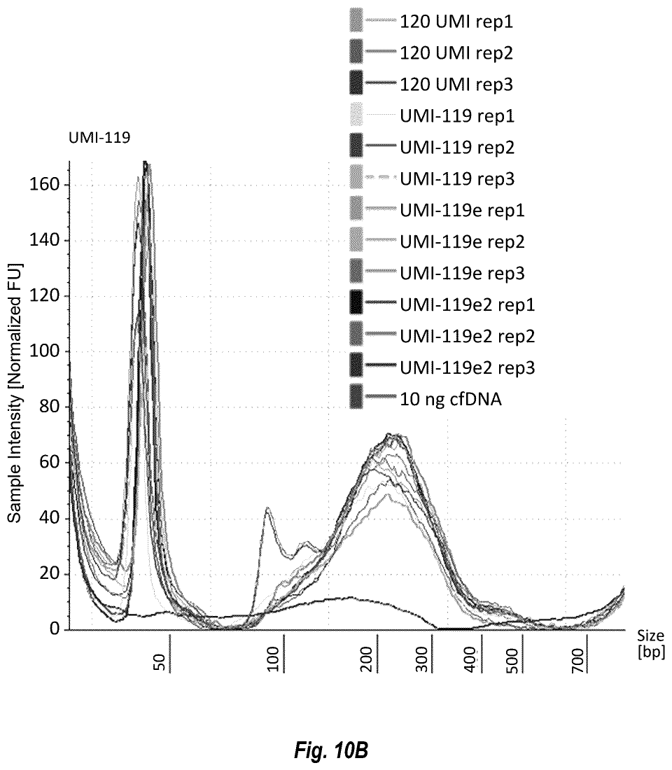

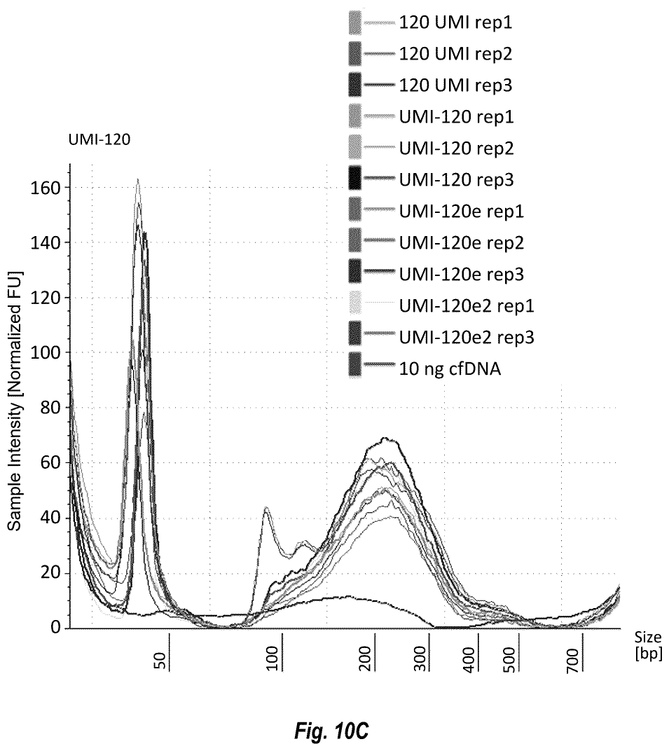

As shown in the plot of FIG. 8, and FIGS. 10A-10C, there was no significant shift in the library size when comparing the improved UMIs to the original sequences.

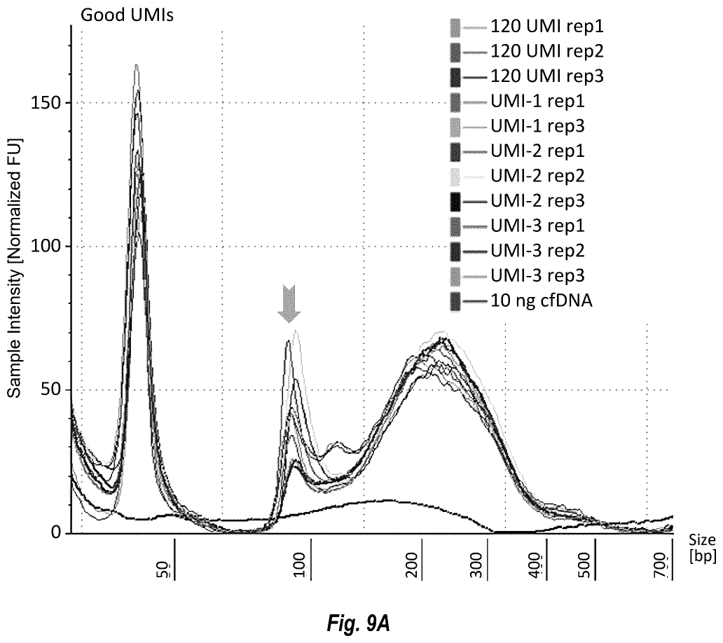

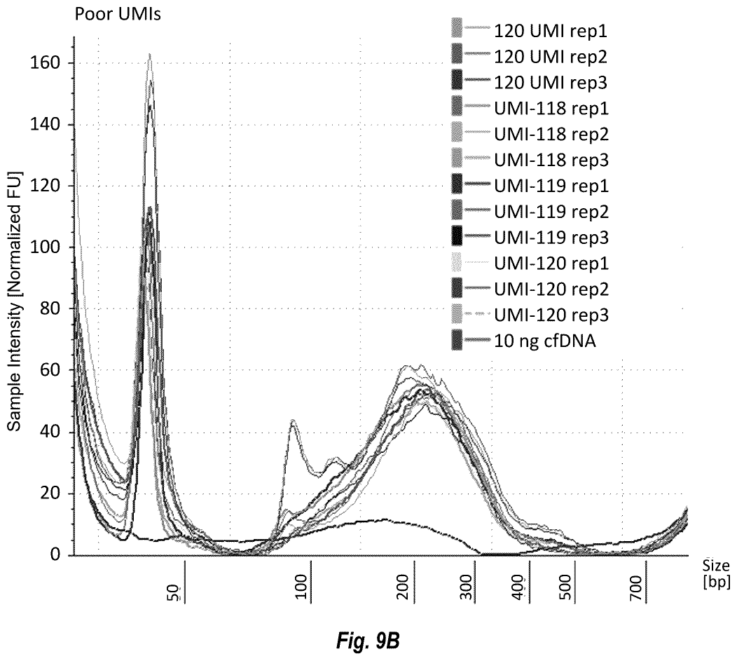

Analysis of the library makeup of the samples after using Good or Poor UMIs revealed that Good UMIs had a similar or higher adapter dimer content compared with the pool of 120 UMIs (as shown by the arrow in FIG. 9A), whereas Poor UMIs exhibited a lower adapter dimer content compared with the pool of 120 UMIs (FIG. 9B). FIGS. 10A-10C show the library makeup for each of the 3 worst performing UMIs in FIG. 9B broken up by UMI (UMI-118, UMI-119, and UMI 120).

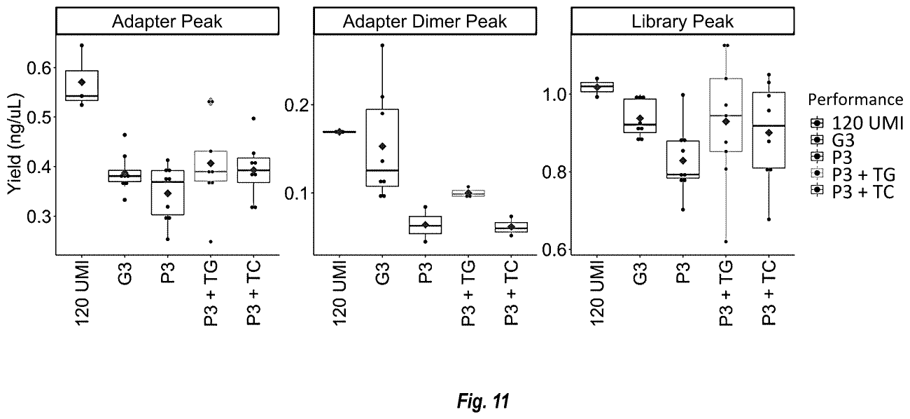

FIG. 11 shows yield for the adapter, adapter dimer and library peaks for the pool of 120 UMIs (120 UMI), the top 3 performing UMIs (G3), the 3 worst performing UMIs (P3) and the 3 worst performing UMIs extended with TG (P3+TG) or TC (P3+TC). A similar free adapter content was observed for good, poor, and extended poor UMIs. Good UMIs formed more adapter dimers than poor UMIs (with and without extensions). And Poor UMIs formed less adapter dimers than the pool of 120 UMIs. Additionally, adapter dimer yield correlated with library yield and ligation efficiency. As shown by data, improving poor performing UMIs with TG- or TC-extension sequences resulted in increased library yield without a substantial shift in library size.

### Example 4. Library Production and Sequencing Using Extended UMIs

The steps of library preparation are shown in FIG. 1A. A library of template nucleic acids is prepared from a sample comprising source nucleic acids prior to enrichment and/or sequencing. The sample preparation includes a fragmentation step that breaks the larger nucleic acid molecules into smaller fragments that are more amenable to next generation sequencing technologies, creating fragmented source nucleic acids. Adapters comprising the UMIs described herein are then attached to the ends of the fragmented source nucleic acids, which can be accomplished by DNA end repair followed by adapter ligation, or by using a transposome system to produce a double-stranded nucleic acid library.

After library production, library fragments are amplified by PCR. Next, the amplified library is added to a solid support, e.g., a flowcell and library clustering and sequencing is carried out sequenced according to any suitable sequencing methodology, such as direct sequencing, including sequencing by synthesis (SBS), sequencing by ligation, sequencing by hybridization, or nanopore sequencing.

Sequencing data are generated and then analyzed using the Illumina DRAGEN™ (Dynamic Read Analysis for GENomics) Bio-IT Platform (or similar analysis platforms). Sequenced reads are aligned to a reference genome or transcriptome. Then, reads at each unique alignment location are independently deduplicated based on the UMI sequences. Analysis of the UMIs described herein is then implemented to screen for and correct errors and quantify unique reads.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A method for sequencing source nucleic acid molecules from a sample using a pool of adapters, wherein the adapters each comprise a unique molecular identifier (UMI), wherein each UMI comprises an oligonucleotide sequence comprising the sequence 5′-X_{N}YZ-3′, wherein the 3′ end of the UMI ends with YZ, wherein YZ does not correspond to GA, GC, or GG, wherein N is an integer greater than or equal to 1 and each X is the same or different, wherein each adapter is attached to a solid support, and wherein each UMI is an oligonucleotide sequence that can be used to identify an individual molecule of a source nucleic acid fragment in the sample, comprising:
a) fragmenting source nucleic acid molecules into nucleic acid fragments;
b) applying the pool of adapters to source nucleic acid fragments in the sample;
c) amplifying both strands of the source nucleic acid-adapter products to obtain a plurality of amplified polynucleotides;
d) sequencing the plurality of amplified polynucleotides, thereby obtaining a plurality of reads each associated with a UMI;
e) identifying a plurality of UMIs associated with the plurality of reads;
f) determining sequences of the source nucleic acid fragments in the sample using the plurality of reads obtained in (c) and the plurality of UMIs identified in (d).

2. A method for sequencing source nucleic acid molecules from a sample using a pool of adapters, wherein the adapters each comprise a primer, a primer binding site, an index sequence, and/or a barcode, wherein each adapter comprises an oligonucleotide sequence comprising the sequence 5′-X_{N}YZ-3′, wherein the 3′ end of the adapter ends with YZ, wherein YZ does not correspond to GA, GC, or GG, wherein N is an integer greater than or equal to 1 and each X is the same or different, wherein each adapter comprises an oligonucleotide sequence comprising the sequence 5′-X_{N}YZ-3′, wherein the 3′ end of the adapter ends with YZ, wherein YZ does not correspond to GA, GC, or GG, and wherein N is an integer greater than or equal to 1 and each X is the same or different, and wherein each adapter is attached to a solid support, comprising:
a) fragmenting source nucleic acid molecules into nucleic acid fragments;
b) applying the pool of adapters to source nucleic acid fragments in the sample;
c) amplifying both strands of the source nucleic acid-adapter products to obtain a plurality of amplified polynucleotides; and
d) sequencing the plurality of amplified polynucleotides.

3. The method of claim 1 or 2, wherein the source nucleic acid is double-stranded.

4. The method of claim 3, wherein the double-stranded source nucleic acids are double-stranded DNA.

5. The method of claim 3, wherein the double-stranded source nucleic acids are ctDNA.

6. The method of claim 3, wherein the double-stranded source nucleic acids are cfDNA.

7. The method of claim 3, wherein the double-stranded source nucleic acids are RNA.

8. The method of claims 3-7, wherein the adapters each comprise a unique molecular identifier (UMI), wherein a first UMI is on the first strand of the double-stranded source nucleic acid fragments.

9. The method of claim 8, wherein the adapters each comprise a unique molecular identifier (UMI), wherein a first copy of the first UMI is on the first strand and a second copy of the first UMI is on the second strand of the double-stranded source nucleic acid fragments.

10. The method of any one of claims 3-9, wherein the adapters each comprise a unique molecular identifier (UMI), wherein a first UMI is on a first strand of the double-stranded source nucleic acid fragments, and a second UMI is on the second strand of the double-stranded source nucleic acid fragments.

11. A method of sequencing a double-stranded nucleic acid library produced by the method of any one of claims 1-10, wherein the adapters each comprise a unique molecular identifier (UMI), and wherein the UMIs are sequenced to provide increased sensitivity in DNA sequencing.

12. A pool of adapters, wherein the adapters each comprise a unique molecular identifier (UMI), wherein each UMI comprises an oligonucleotide sequence comprising the sequence 5′-X_{N}YZ-3′, wherein the 3′ end of the UMI ends with YZ, wherein YZ does not correspond to GA, GC, or GG, wherein N is an integer greater than or equal to 1 and each X is the same or different, and wherein each adapter is attached to a solid support.

13. A pool of adapters, wherein the adapters each comprise a primer, a primer binding site, an index sequence, and/or a barcode, wherein each adapter comprises an oligonucleotide sequence comprising the sequence 5′-X_{N}YZ-3′, wherein the 3′ end of the adapter ends with YZ, wherein YZ does not correspond to GA, GC, or GG, wherein N is an integer greater than or equal to 1 and each X is the same or different, and wherein each adapter is attached to a solid support.

14. The pool of adapters of claim 12 or 13, wherein each N is an integer from 1 to 40, 1 to 30, 1 to 20, or 1 to 10.

15. The pool of adapters of any one of claims 12-14, wherein each N is 2, 3, 4, 5, 6, 7, 8, 9, 10, 11, 12, 13, 14, 15, 16, 17, 18, 19, 20, 21, 22, 23, 24, 25, 26, 27, 28, 29 30, 31, 32, 33, 34, 35, 36, 37, 38, 39, or 40.

16. The pool of adapters of any one of claims 12-15, wherein each N is an integer from 2 to 7.

17. The pool of adapters of any one of claims 12-15, wherein each N is an integer from 4 to 7.

18. The pool of adapters of any one of claims 12-15, wherein each N is an integer from 4 to 5.

19. The pool of adapters of any one of claims 12-15, wherein each N is an integer from 6 to 7.

20. The pool of adapters of any one of claims 12-19, wherein YZ corresponds to TX.

21. The pool of adapters of any one of claims 12-20, wherein YZ corresponds to TA, TC or TG.

22. The pool of adapters of any one of claims 12-21, wherein the solid support is a flowcell.

23. The pool of adapters of any one of claims 12-22, wherein the adapters comprise DNA.

24. The pool of adapters of any one of claims 12-23, wherein the adapters comprise RNA.

25. The pool of adapters of any one of claims 12-24, wherein the adapters comprise an RNA: DNA hybrid.

26. The pool of adapters of any one of claims 12-25, wherein the adapters are methylated.

27. The pool of adapters of any one of claims 12-26, wherein the adapters are single stranded.

28. The pool of adapters of any one of claims 12-26, wherein the adapters are double stranded.

29. The pool of adapters of claim 28, wherein the double stranded adapters each comprise a UMI, wherein the UMI is on only one strand.

30. The pool of adapters of claim 28, wherein the double stranded adapters each comprise a UMI, wherein the UMI is on both strands.

31. The pool of adapters of any one of claims 12-30, wherein each adapter in the pool of adapters comprises a UMI and wherein the UMI is a unique UMI shared by no other adapter in the pool of adapters.

32. The pool of adapters of any one of claims 12-30, wherein each adapter in the pool of adapters comprises a UMI and wherein more than one adapter in the pool of adapters has the same UMI, but wherein that UMI differs from other adapters in the pool of adapters.

33. The method of any one of claims 1-11 or the pool of adapters of any one of claims 12-32, wherein the adapters each comprise a primer.

34. The method of any one of claim 1-11 or 33 or the pool of adapters of any one of claims 12-33, wherein the adapters each comprise a primer binding site.

35. The method of any one of claim 1-11 or 33-34 or the pool of adapters of any one of claims 12-34, wherein the adapters each comprise an index sequence.

36. The method of any one of claim 1-11 or 33-35 or the pool of adapters of any one of claims 12-35, wherein the adapters each comprise a barcode.

37. The method of any one of claims 1-11, wherein the method uses the pool of adapters of any one of claims 12-36.
